(19) United States
(12) Patent Application Publication
(10) Pub. No.: US 2018/0200293 A1
(43) Pub. Date: Jul. 19, 2018

(54) STABILIZED GOLD NANOPARTICLES

(71) Applicant: Khashayar Ghandi, Sackerville, NB (CA)

(72) Inventor: Khashayar Ghandi, Sackerville, NB (CA)

(21) Appl. No.: 15/322,619

(22) PCT Filed: Jun. 30, 2015
(86) PCT No.: PCT/IB2015/002035

§ 371 Date: Dec. 28, 2016

Related U.S. Application Data
(60) Provisional application No. 62/019,049, filed on Jun. 30, 2014.

(51) Int. Cl. A61K 33/24; C12Q 1/04; A61K 41/00; A61K 9/14; B01J 23/52; B01J 31/02; B01J 35/00; B01J 37/04

(52) U.S. Cl. CPC A61K 33/24; C12Q 1/04; A61K 41/00; B01J 37/04; B01J 23/52; B01J 31/0271; B01J 35/0013; A61K 9/145

(57) ABSTRACT

The present disclosure relates to gold nanoparticles stabilized with benzylalkyl(C8-C18) ammonium chloride, and methods and uses comprising the same.

### CROSS REFERENCE TO RELATED APPLICATIONS

This application claims priority to U.S. Provisional Patent Application Ser. No. 62/019,049, filed Jun. 30, 2014, and is incorporated herein by reference.

### FIELD

This invention relates to electron capture and oxidation by metal nanoparticles stabilized by certain ionic compounds

### BACKGROUND

The solvated electron in water, e⁻_{aq}, is the most elementary reducing agent(1), and along with its precursors is implicated in various chemical and biological processes, including DNA and cell damage during radiation therapy(2). It can react with various species to form radicals, for example it can react with oxygen to form a superoxide radical. These radicals in biological systems cause oxidative stress, and cell damage. In chemical reactions, these solvated electrons, and their resulting radicals can act reducing agents for a variety of known chemical processes.

Thus, in order to control their effects, various scavengers have been found. Most of these scavengers, are free radical scavengers, and thus have limited effect in capturing the pre-solvated and solvated electrons in solution before they form radical species. Other scavengers such as KNO₃, DMSO, Isopropanol etc. have been used to capture the pre-solvated electron, however they are required to be used in high concentrations (of upto 2 Molar)(3), and would not entirely be suitable for biological applications, or all chemical applications, as these scavengers might interfere in an undesirably manner with the chemical reactants. Thus there is a need to identify, and develop new scavenging agents capable of fast capture of the solvated electron, and its precursors (the pre-solvated electron), at low scavenger concentrations, that is preferably non-toxic.

### SUMMARY

The disclosure provides metal nanoparticles stabilized by an Ionic Liquid (IL)-surfactant and that remains stable in solution for weeks without aggregation. The disclosure also relates to methods and uses comprising the metal nanoparticles.

In a general aspect, the disclosure provides for a solution of metal nanoparticles that is adapted for capture of free electrons from a target or source. In related aspect, the disclosure generally provides a method of capturing free electrons in solution comprising exposing a target or source to a solution of metal nanoparticles.

In another aspect the disclosure relates to a stabilized metal nanoparticle composition comprising metal nanoparticles and a non-covalently bound ligand in a stable single phase aqueous solution. In embodiments, the nanoparticles are monometallic, bimetallic, or polymetallic. In some embodiments, the nanoparticles comprise gold, silver, copper, or titanium nanoparticles, or alloys thereof, and in particular embodiments the nanoparticles are gold nanoparticles.

In further embodiments of the above aspect, the metal nanoparticles may be capped with the non-covalently bound ligand. In particular embodiments, the non-covalently bound ligand is benzylalkyl(C8-18)ammonium chloride.

In other embodiments of the above aspect, the composition includes metal nanoparticles that are capable of capturing pre-solvated electrons from a solution.

In any of the above aspects and embodiments the size of the metal nanoparticles may range from between 1-10, 1-50, 1-100 and 1-1000 nanometers.

In another aspect the disclosure relates to a therapeutic agent for radiation therapy or radiation protection comprising the composition as described herein, and optionally a biologically compatible carrier. In some embodiments, the therapeutic agent is capable of capturing pre-solvated and solvated electrons, and may prevent the formation of reactive oxygen and nitrogen species. In some embodiments, the therapeutic agent is capable of radioprotective effects and may comprise reducing the secondary effects of radiation induced cell damage.

In another aspect the disclosure relates to a topical composition comprising the composition described herein and a carrier, wherein the composition is formulated for topical application. In embodiments, the therapeutic agent is formulated as a lotion, gel, rinse, or cream. In some embodiments, the therapeutic agent may further comprise an additional antimicrobial agent. In embodiments, the topical composition may be used in the manufacture of a medicament for protection from radiation exposure. In embodiments, the topical composition may be sued in the manufacture of a medicament for protection from microbial infection.

In another aspect, the disclosure relates to a catalyst for oxidation reactions comprising the composition as described herein. In embodiments, the catalyst is capable of inhibiting radiation induced chemical reactions. In other embodiments, the catalyst is capable of moderating radiation induced chemical reactions. In related aspects, the catalyst may be used for pre-solvated electron based chemical reactions. In embodiments, the catalyst is capable of inhibiting pre-solvated electron based chemical reactions. In embodiments, the catalyst is capable of moderating pre-solvated electron based chemical reactions. In other related aspects, the catalyst may be used for solvated electron based chemical reactions and in some embodiments, may be capable of inhibiting solvated electron based chemical reactions. In other embodiments the catalyst may be capable of moderating solvated electron based chemical reactions.

In another aspect the disclosure provides a method for preparing a stable single aqueous phase composition comprising metal nanoparticles comprising mixing the metal nanoparticles with an aromatic alkyl ammonium halide. In embodiments of this aspect, the metal nanoparticles in the single aqueous phase are stable in water for at least three months. In some embodiments, the stable single aqueous phase comprises anionic and cationic components, and wherein the metal nanoparticles are catalytically active.

In the various aspects relating to the metal nanoparticles the nanoparticles may comprise one or more general geometrical arrangements. In some embodiments, the shape of the metal nanoparticles is selected from a spherical, a triangle, a square, a rectangle, a rhombus, a diamond, a pyramid, and other polygonal shapes comprising at least 5, 6, 7, 8, 9, 10, 11, 12, 13, 14, 15, 16, 17, 18, 19 or 20 or more sides or faces.

In another aspect, the disclosure relates to a microbial sensor comprising the composition as described herein, wherein the metal nanoparticles are provided in a shape arrangement that provides for a change in one or more of the optical, physical, or chemical properties of the metal nanoparticles upon binding to a microbial organism.

In yet another aspect, the disclosure provides an assay for detecting the presence of a microbe, wherein the assay comprises contacting a sample to be analyzed with an amount of the composition described herein; detecting a change in at least one property of the metal nanoparticles selected from the group consisting of the optical, physical, or chemical properties of the metal nanoparticles, and wherein detecting a change in at least one property of the metal nanoparticles indicates the presence of the microbe in the sample.

As discussed herein, the ability of these nanoparticles to capture electrons in solution with very large rate constants is believed to be unprecedented among known electron scavengers, and provides for a number of methods and uses comprising these nanoparticles. Other aspects and embodiments will be apparent to one of ordinary skill in the art in view of the disclosure that follows.

### DRAWINGS

FIG. 1. Metal nanoparticle stabilization by an alkyl ammonium chloride mechanism (e.g., Astruc and coworkers).

FIG. 2. Imidazolium cation coordination and stabilization schematic

FIG. 3. Proposed schematic for a gold nanoparticle stabilized by TOABr.

FIG. 4. Proposed schematic for a gold nanoparticle stabilized by Bac-14.

FIG. 5. TEM image of as prepared gold nanoparticle. Size distribution is normally distributed. Average diameter is 24 nm

FIG. 6. EDX analysis of Au/bac-14 as prepared in water. The carbon peak from the substrate is omitted to observe nearby peaks.

FIG. 7. UV-visible spectrum depicting surface plasmon band of Au/bac-14 AuNPs synthesized by a single-phase process described above. Surface plasmon band is observed with max wavelength at 520 nm.

FIG. 8: ¹H NMR of 0.01 M solution of Bac-14 in D₂O.

FIG. 9: ¹H NMR of 0.01 M solution of AuNP/Bac-14 in D₂O.

FIG. 10: ¹³C NMR of 0.01 M solution of Bac-14 in D₂O.

FIG. 11: ¹³C NMR of 0.01 M solution of AuNP/Bac-14 in D₂O.

FIG. 12. FT-IR spectra of Bac-14 and AuNP.

FIG. 13. Crystal structure of Bac-14.

FIG. 14. Structure of Bac-14 and Cl-stabilizing a Au(0) atom optimized with DFT/LANL2DZ conditions.

FIG. 15. Sites of Muonium addition in bac-14.

FIG. 16A-B. (A) Transverse Field μSR spectrum of bac-14 in water at 274 K, showing two free radicals with muon hfcc of 428(2) MHz and 349(3) MHz. The diamagnetic peak is much larger than the two free radical peaks. (B) The calculated spin density of the cyclohexadienyl radical (left) and bac-14 radical (right). Molecular geometries were optimized at the B3LYP/6-31G* level of theory. The hfcc of the cyclohexadienyl radical is ˜515 MHz(36), while the calculated muon hfcc of the para bac-14 radical at 0 K is 441.024 MHz. The calculated hfcc of the meta bac-14 radical at 0 K is 455 MHz. For the ipso bac-14 radical at 0 K it is 409 MHz.

FIG. 17A-B. TF spectra, showing a diminished diamagnetic fraction for the aqueous solution of bac-14. The disparity in the diamagnetic fraction is due to the presence of paramagnetic species, as outlined in Reactions 1, 3, and 4. These are either muoniated radicals or Mu. Superior peaks (red) represent aqueous solutions of the cationic surfactant at 298 K and an applied field H, 1000 G (A) and 330 K at the same field (B). Inferior peaks (blue) represent aqueous solutions of AuNPs at equivalent temperatures and fields. Fourier amplitudes (FA) are given with uncertainty in parentheses.

FIG. 18. Calculated rate constant (10¹⁵ M⁻¹ s⁻¹) of electron-nanoparticle reaction based on a modified Stokes-Einstein kinetic form. Rate constants scale linearly with particle size (nm).

FIG. 19A. Aggregation of gold nanoparticles in the presence of E. coli, S. aureus, and S. Cerevisiae. Upon addition of a colored solution of gold NPs (which is purple in a colored form of the depicted illustration) to the microbes in aqueous solution, the gold NPs bind and aggregate with the microbes forming a precipitate (also purple in color). In the absence of any microbe, the gold NPs remain stable in solution (and color the solution purple).

FIG. 19B. Depicts aggregation of gold nanoparticles with E Coli.

FIG. 19C. Depicts E Coli in the absence of added gold nanoparticles.

### DETAILED DESCRIPTION

All the scientific terms herein are used in connection with their standard scientific meaning. All techniques and reagents described herein, unless otherwise described, are standard and generally known in the art.

The metal nanoparticles, of the present invention are synthesized using a green method to make stable metal nanoparticles in a single aqueous phase that are non-covalently stabilized by an aromatic alkyl ammonium halide. They may additionally be synthesized by various methods known to one well versed in the art.

In this patent, we present novel gold nanoparticles (AuNPs) stabilized by an Ionic Liquid (IL)-surfactant in solution for weeks without aggregation. We also report large rate constants, unprecedented among known electron scavengers, for electron transfer to these gold nanoparticles (AuNPs) with diameter ˜25 nm in aqueous solutions, ke=2×10¹⁵ M⁻¹ s⁻¹. The IL used-benzyldimethyltetradecylammonium chloride (Bac-14)-stabilizes the NPs non-covalently, but rather via electrostatic interactions. Long-term stabilization of AuNPs by a quaternary ammonium IL has not been described in the literature to our knowledge (4, 5)

Astruc et. al. proposed a model based on differential anion stabilization of iridium NPs in the following order: polyoxometallate>citrate>polyacrylate˜chloride (FIG. 1). They argue that the anion must be at the surface of the metal, because the stabilization of the NPs correlates with the sterics of the anion(5).

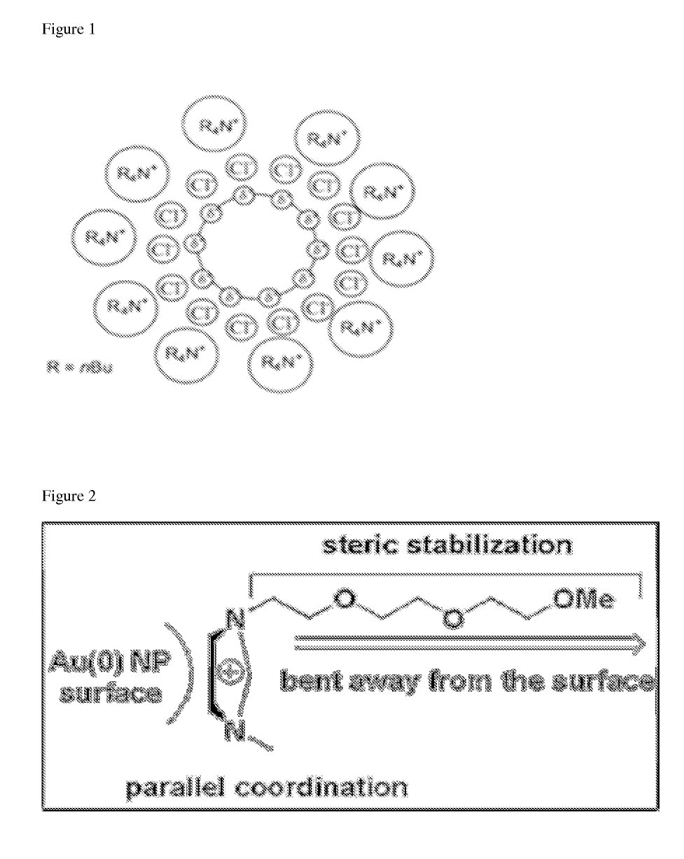

However, some mechanistic descriptions of AuNP stabilization by imidazolium chloride ILs posit that the cation is at the surface of the metal (FIG. 2)(6).

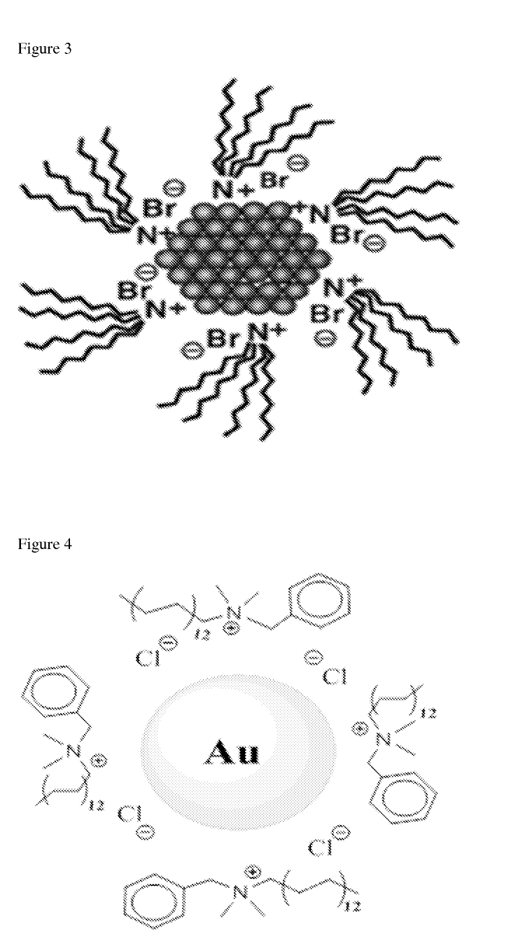

This conformation around the gold nanoparticles was proposed by surface-enhanced Raman spectroscopy (SERS) studies. Here, we propose stabilization, for our nanoparticles similar to what was proposed by Thomas et. al. which they based on the strong association of photo-responsive molecules on the surface of gold nanoparticles (FIG. 3)(4, 7). Our proposed mechanism of stabilization is outlined in FIG. 4.

Formation of AuNPs was determined by UV-Vis and TEM and conformational assignment of the Bac-14-stabilized AuNPs was based on FT-IR, ¹H and ¹³C NMR, and computational study.

Elucidation of the stabilization mode of these nanoparticles can provide information on the viability of these particles as catalysts for oxidation reactions. There have been numerous studies demonstrating the promise of Au(0) as a catalyst, whether it is in varying nanoparticle size, one-dimensional sheet form or simply as gold powder(8-12).

Also, considering that eₚᵣₑ⁻ can contribute significantly to DNA damage (3)(3), these AuNPs could have several potential therapeutic applications as electron scavengers in radiobiological processes. AuNPs could act as general, efficient and fast electron scavengers for various radiation chemistry processes, as the result of a large electron-scavenging rate (2×10¹⁵ M⁻¹ s⁻¹) that is significantly higher than that of many currently known electron scavengers at significantly lower concentrations. The compatibility of AuNPs with synthetic inorganic materials as well as biological systems indicates a potential to incorporate Au/bac-14 as an extremely efficient electron acceptor in to a variety of environments.

The metal nanoparticles disclosed herein may adopt various geometrical configurations that can change in response to one or more applied external stimulus. The changes in configuration that are induced and/or caused by an external stimulus or input can provide for a change in one or more of the optical, physical, or chemical properties of the metal nanoparticles and which can be detected. Thus, in such aspects, the metal nanoparticles can be used in as sensors and/or in assays and method for the detection of one or more particular agent(s). For example, in some embodiments the metal nanoparticles can bind to a microbe and, upon binding, change conformation to produce an observable change in the optical, physical, and/or chemical properties of the nanoparticles. As such, embodiments of the disclosure provide for sensors comprising the metal nanoparticles that can detect the presence of an agent such as, for example, a microbe and/or a source of radiation. In such embodiments, the assay may be performed by contacting (e.g., either directly (i.e., for a microbe) or indirectly contacting (i.e., for a source of radiation)) the nanoparticles with a sample to be assayed. In embodiments relating to the detection of a microbe, the sample may be an organic sample (e.g., biological) or non-organic (e.g., work surface) and can comprise a biological culture that is propagated from a remote source (e.g., bacterial culture from a sample swab of a work surface). The sensors and assays may also comprise a control that comprises a source that does not include a microbe or source of radiation energy.

In embodiments relating to microbial sensors, the nanoparticles disclosed herein comprise an active surface having binding affinity for outer membranes of microbial organisms. Upon binding, the nanoparticles can form larger particles (aggregates) which provide for observable changes in the optical (color, light absorbance, etc.), physical, and/or chemical properties that are dependent on particle size and chemical microenvironment. In some embodiments, the binding of nanoparticles with a microbe reduces the stability on the particles in solution, causing aggregation of the nanoparticles which results in an observable change such as, for example, the color of solution (e.g., from colored to colorless) and/or formation of precipitate.

### Uses and Methods

In aspects, the nanoparticles of the present disclosure are useful in the capture of free electrons in solution by exposing the target to a solution of metal nanoparticles. In other aspects, the nanoparticles of the present disclosure are useful in the capture and transfer of free electrons emitted and/or released from matter that is impacted by a radiation source, and thereby generate electrical energy in solution by exposing the target to a solution of metal nanoparticles

In one embodiment, the present disclosure includes metal nanoparticles, optionally with a biocompatible carrier that can act as therapeutic agents or drugs for radiation therapy, to prevent secondary radiation damage to cells.

In another embodiment, the present disclosure includes metal nanoparticles, optionally with a biocompatible carrier, that can act as therapeutic agents or drugs capable of capturing pre-solvated and solvated electrons, to prevent the formation of reactive oxygen and nitrogen species.

In another embodiment, the nanoparticles of the present disclosure can act as catalysts capable of acting as a catalyst for oxidation reactions.

In another embodiment, the nanoparticles of the present disclosure can act as catalyst capable of inhibiting, or moderating radiation induced chemical reactions.

In another embodiment, the nanoparticles of the present disclosure can act as catalyst capable of inhibiting, or moderating for pre-solvated electron based chemical reactions.

In another embodiment, the nanoparticles of the present disclosure can act as catalyst capable of inhibiting, or moderating for solvated electron based chemical reactions.

In another embodiment, the nanoparticles of the present disclosure can act as a stable suspension of metal nanoparticles, in a single aqueous phase, bound electrostatically to an aromatic alkyl ammonium halide, capable of acting as a metal catalyst, for chemical reactions.

In some aspects and embodiments, the disclosure provides for compositions and formulations that comprise the metal nanoparticles. The compositions comprising the metal nanoparticles disclosed herein may be formulated as solutions and emulsions. Such formulations may be used in methods of treatment comprising radiation therapy and/or in methods for protection from radiation (e.g., protection from exposure to a source of radiation). Any suitable excipient or combinations of excipients may be used in the compositions and formulations including, for example, emulsifiers, surfactants, stabilizers, dyes, penetration enhancers and anti-oxidants. Suitable carriers can be added in the compositions and can include non-limiting examples of, water, salt solutions (e.g., buffers), alcohols, polyethylene glycols, gelatine, lactose, magnesium sterate and silicic acid. The compositions may be sterile or non-sterile aqueous or non-aqueous solutions and/or emulsions. The compositions can also be formulated as suspensions in aqueous, non-aqueous or mixed media. Aqueous suspensions may further contain substances that increase the viscosity of the suspension and may also contain stabilizers. The solutions may also contain buffers, diluents and other suitable additives. The compositions can include other adjunct components that are compatible with the metal nanoparticles. In some embodiments, the compositions may be formulated and used as foams, emulsions, microemulsions, lotions, gels, mousses, creams, and jellies. In some embodiments, the compositions and formulations can comprise an additional antimicrobial agent in addition to the metal nanoparticles. Such antimicrobial agents are generally known in the art.

The nanoparticles disclosed may be used to inhibit, treat, or prevent, microbial infection in a subject (e.g., an animal, mammalian, or human subject). For such uses and methods, the nanoparticles may be provided as a composition of matter that comprises the metal nanoparticles and at least one auxiliary that is used in the art of formulation/formulary sciences such as, for example, extenders (e.g., solvents or solid carriers) or surface-active compounds (e.g., surfactants).

In some aspects and embodiments, the disclosure provides for the use of the nanoparticles described herein for the manufacture of a formulation or medicament for preventing microbial infection. In other aspects and embodiments, the use of the nanoparticles may be for the manufacture of a formulation or medicament for radiation protection (e.g., protection from exposure to radiation). In yet further aspects and embodiments, the use of the nanoparticles described herein may comprise a therapy comprising radiation treatment.

In other embodiments, the disclosure provides for methods and assays that are effective for detecting the presence of a microbe in a sample. The sample may be a sample as discussed above. In embodiments, the methods and assays may comprise one or more control samples that provide a relative measurement for a sample that lacks the presence of any microbe.

All references cited herein are incorporated by reference.

While certain embodiments are provided in the Examples below, one of ordinary skill in the art will appreciate that the Examples and Figures provided herein are merely illustrative and do not limit the appended claims. Other embodiments and aspects are within the scope of the disclosure.

### EXAMPLES

### Example 1—Preparation of the Gold Nanoparticles (AuNp) Using Benzyldimethyltetradecylammonium Chloride

Hydrogen tetrachloroaurate(III) trihydrate (HAuCl₄.3H₂O) was purchased from Alfa Aesar. Benzyldimethyltetradecylammonium chloride (bac-14) was purchased from TCl America. Sodium Borohydride was purchased from Sigma Aldrich. All chemicals were purchased and used without further treatment. All reagents used were spectroscopic grade or better. Deionized water used in all syntheses was obtained from a Millipore Milli-Q, with resistivity of 18.2 MΩ·cm at 25° C.

The synthesis was carried out as follows. Aqueous HAuCl₄ (0.1 M, 25 mL) (Alfa Aesar) was stirred at 40° C. under a reduced atmosphere of Nitrogen for 15 minutes. A solution of Bac-14 (4 g, 10 mmol) in 80 mL water was prepared and added to the aqueous HAuCl₄. A separate solution of NaBH₄ (800 mg, 21 mmol) in 25 mL of distilled deionized water was then added dropwise to the reaction mixture over several minutes. Reduction was instantaneous and the mixture was allowed to stir under mild heat for 2-3 hours.

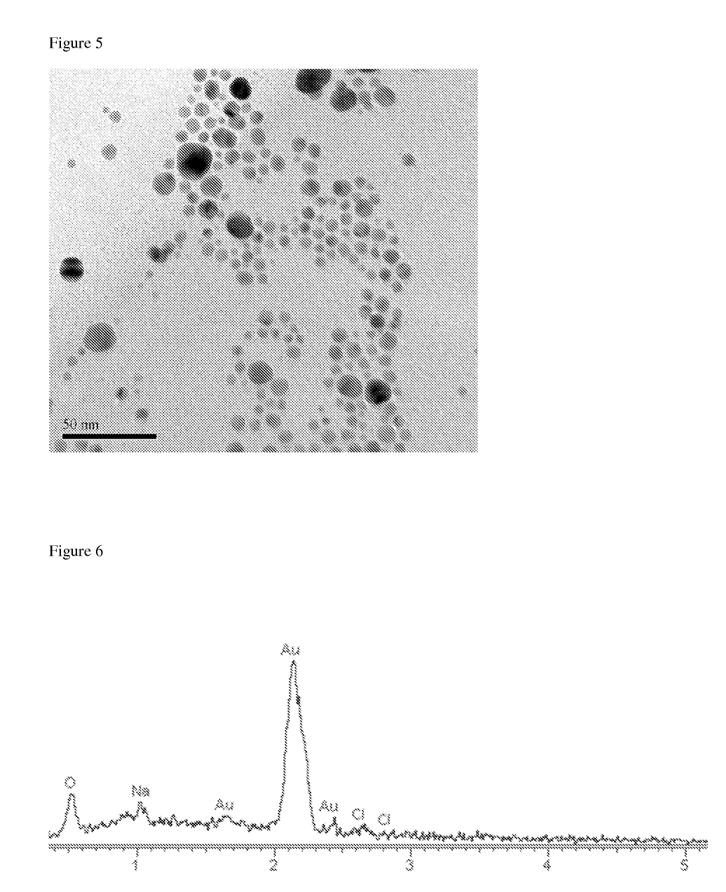

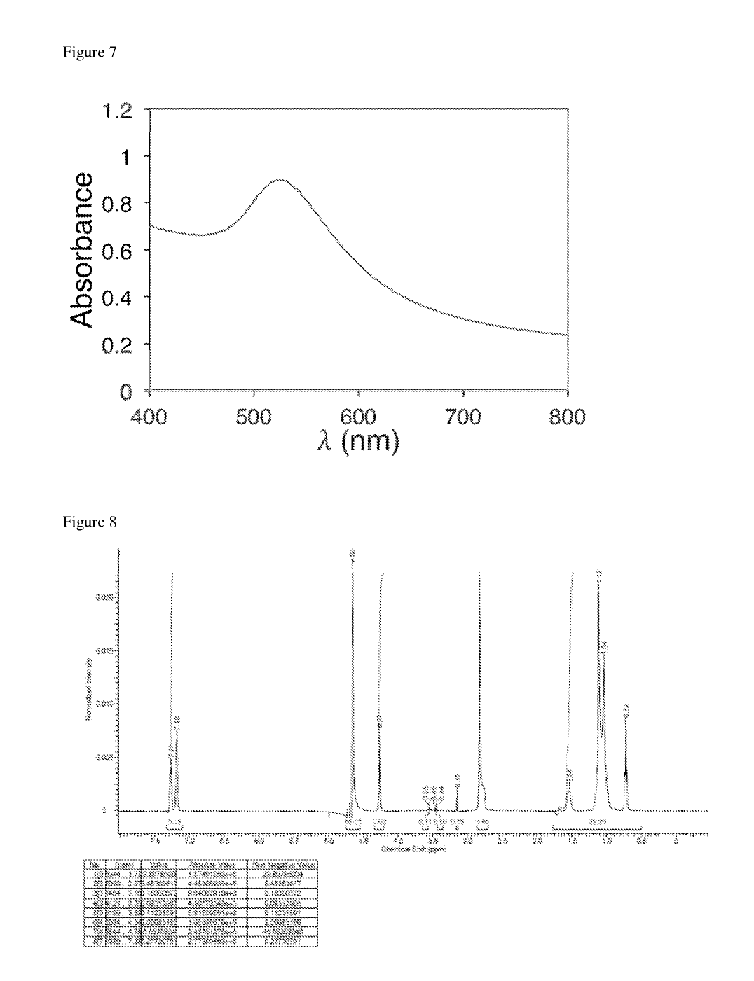

The nanoparticles were then characterized by a scanning/transition electron microscope (JEOL 2011 STEM, operating at 200 kV, combined with energy disperse X-ray spectroscopy (EDX) detection). The TEM and EDX spectra of the nanoparticles are presented in FIGS. 5 and 6 respectively. The surface plasmon resonance of solution-based AuNPs was measured using a Cary-100 UV-Vis spectrometer with maximum absorbance observed near 520 nm (FIG. 7). All samples submitted for UV-Vis spectroscopy were diluted by a factor of 1/100. Attenuated total reflectance (ATR) spectroscopy was performed using a Thermo Scientific Nicolet iS5 spectrometer equipped with an iD5 ATR accessory (example 4).

### Example 2—Preparation of the Gold Nanoparticles Using Benzyl Dimethyl Alkyl (C12, C16 and C18) Ammonium Chloride

The synthesis of these nanoparticles was carried out according to the procedure in example 1, where the Benzyldimethyltetradecylammonium chloride (bac-14) surfactant was replaced by the surfactants Benzyldimethyldodecylammonium chloride (C12), Benzyldimethylhexadecylammonium chloride (C16), and Benzyldimethyloctadecylammoniumchloride (C18) at an 8 mmol concentration. These nanoparticles were characterized by UV/Vis spectroscopy, and the surface plasmon band peaks are presented in table 1.

### Example 3—Characterization with Nuclear Magnetic Resonance (NMR) Spectroscopy

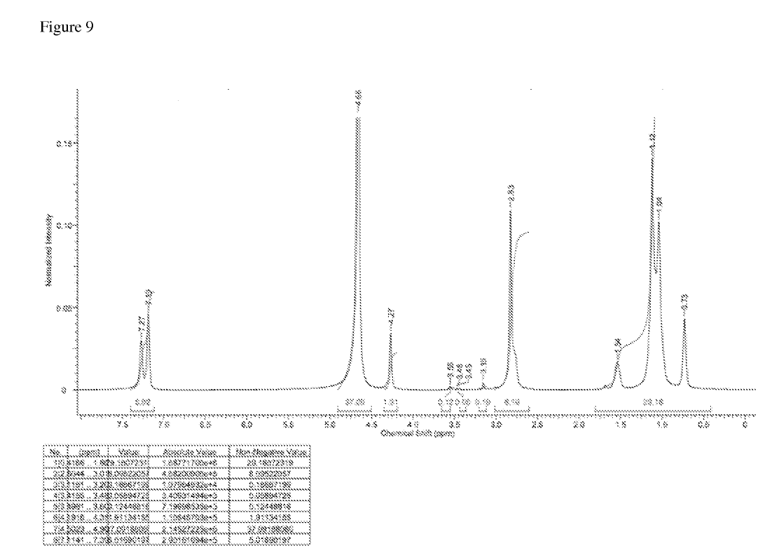

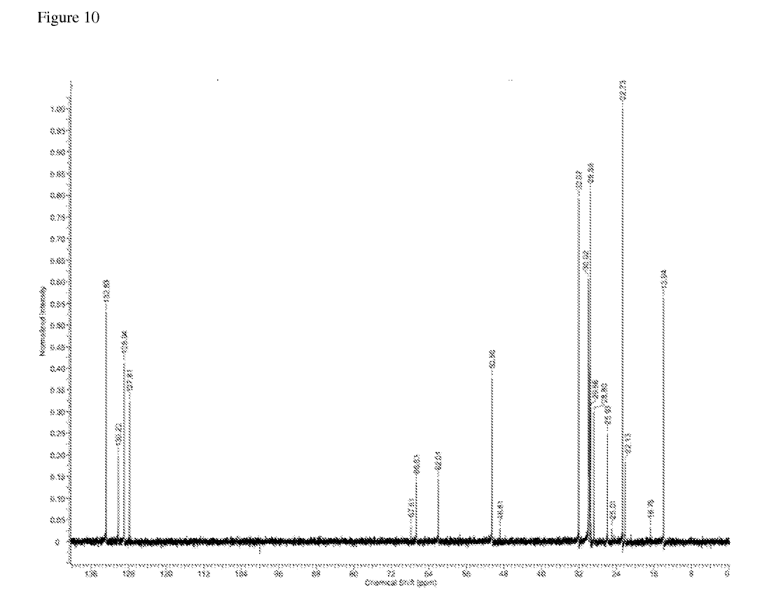

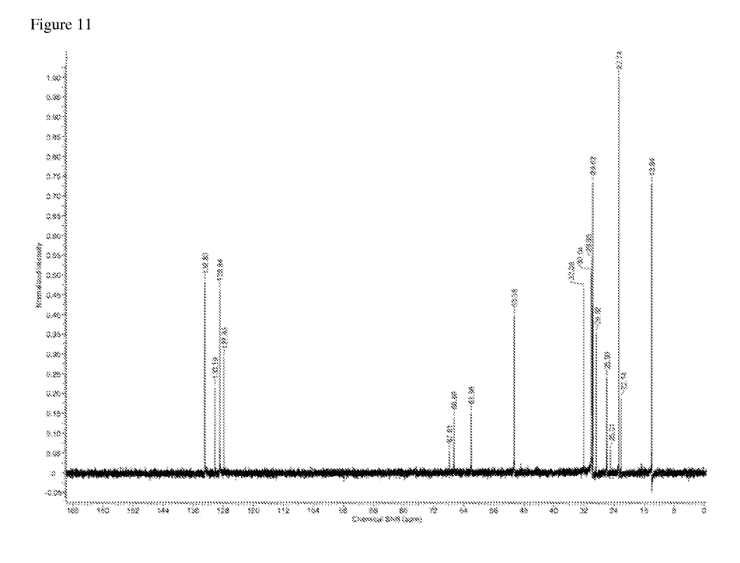

The ¹H and ¹³C NMR spectrums (FIG. 8-11) of Bac-14 and the gold nanoparticle solution of example 1, demonstrated that Bac-14 remains virtually chemically identical when stabilizing the AuNPs, therefore there is no NMR-detectable covalent interaction between Bac-14 and the Au(0).

### Example 4—Characterization with Fourier Transform Infra-Red (FTIR) Spectroscopy

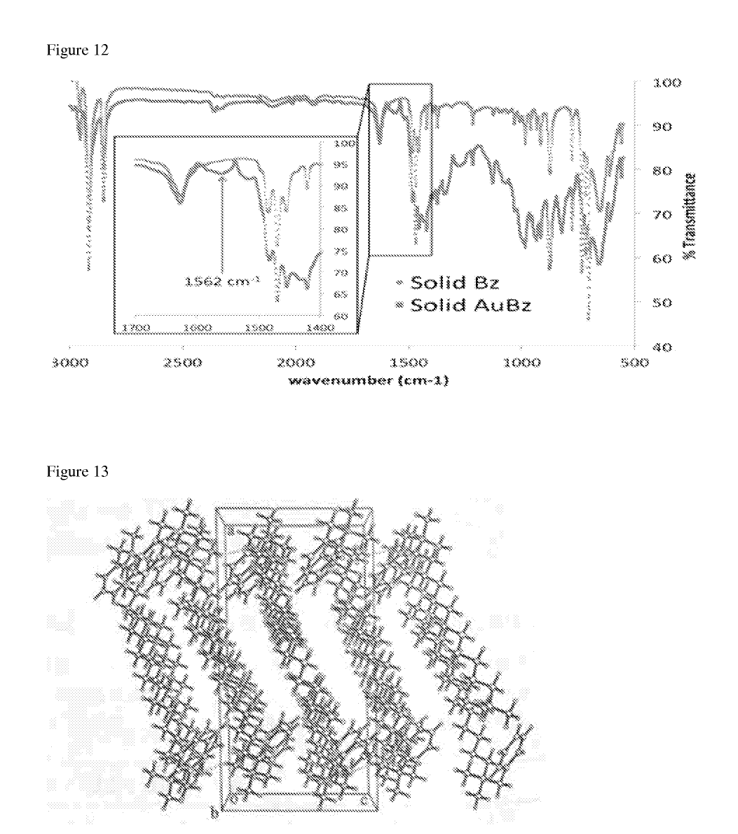

The IR spectra, however, showed some significant differences between pure Bac-14 and the dried stabilized AuNPs. The most significant change outside the fingerprint region was a new peak at 1562 cm⁻¹ in the AuNP IR (FIG. 12). A computational assessment was performed on a single Bac-14 molecule stabilizing one Au(0) and one Cl⁻ atom using the density functional theory (DFT) method and the LANL2DZ basis set(13-15). This basis set is widely used in studying compounds and clusters containing heavy elements and is routinely employed in the framework of DFT calculations(16). The optimized structure depicted in FIG. 13 yielded a vibration (1547 cm⁻¹) in good correspondence with the new signal in the IR spectrum. The calculated coordinates of the optimized structure is presented in table 2.

The changes seen in the fingerprint region has been attributed to the lengthy alkyl chain gaining more vibrational freedom while in a stabilizing conformation. If Bac-14 is surrounding the AuNP in a radial arrangement similar to a micelle (with the alkyl tails pointing outwards as seen in FIG. 3) then they have much more freedom sterically as opposed to the close stacking of solid Bac-14, as can be seen in its crystal structure (FIG. 13).(17)

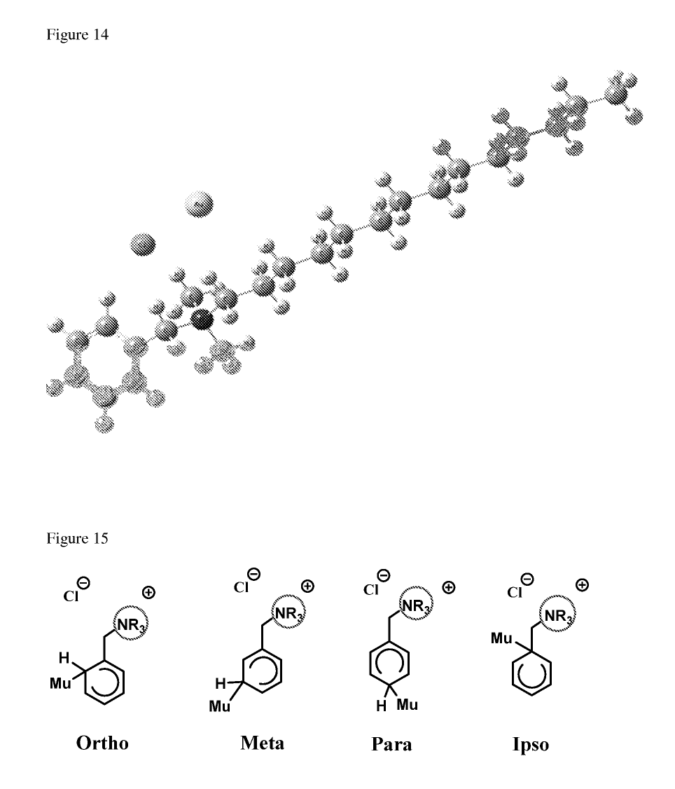

The optimized structure displayed in FIG. 14 demonstrates that the chloride and cationic head of Bac-14 are both close to the Au(0) atom in a minimum energy structure.

### Example 5—Characterization with Muon (μSR) Spectroscopy

To deconstruct the complex electron-transfer reactions in water, we used a highly sensitive spectroscopic technique involving the positive muon (μ+) using the muon spin resonance (μSR) technique (18). The μSR technique is a type of spin spectroscopy that makes use of short-lived subatomic particles, muons (μ⁺), which are sensitive magnetic and electronic probes of matter.

A degassed 0.1 M aqueous solution of the dissolved surfactant (bac-14) was used as a reference. In the absence of the AuNPs the charged surfactant in the reference solution was unable to effectively trap the solvated or pre-solvated electrons. These electrons therefore combine with positive muons or MuH₂O⁺ ions to form paramagnetic muonium, an ultra-light hydrogen atom (0.11H) that adds to the aromatic ring of the surfactant (FIG. 15).

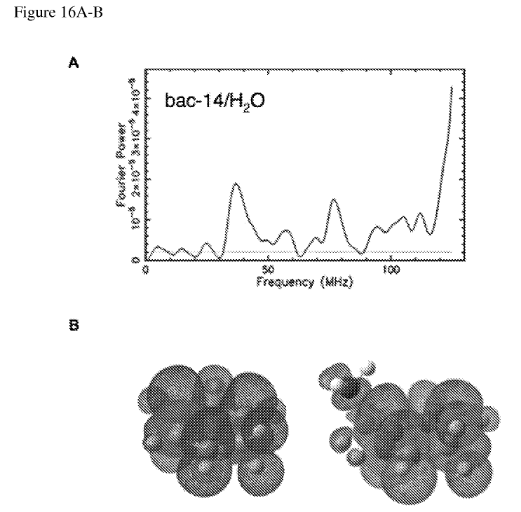

The addition products (radicals) observed in the μSr spectrum (FIG. 16), are identified through the calculation of the isotropic Fermi contact coupling of the different nuclei for muonium addition across the aromatic double bonds. The results for the ortho addition (FIG. 15) agree well with the experimentally observed radical at 425 MHz, and the ipso radical assigned to the observed radical at ˜350 MHz (table 3).

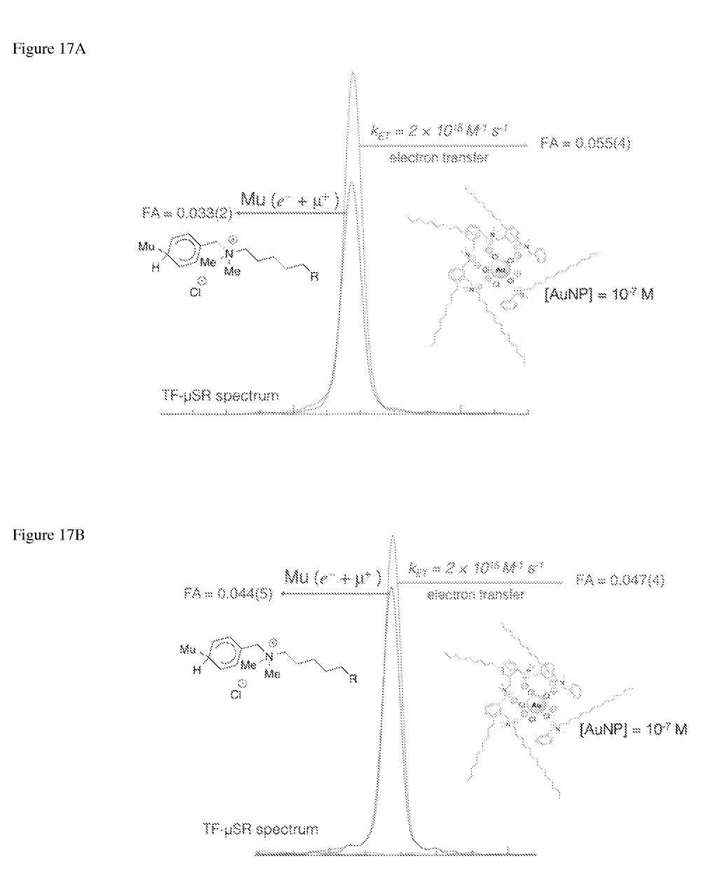

Experimental data for the aqueous AuNP/bac-14 shows that the electrons in solution are directly affected by the addition of the AuNPs. The diamagnetic fraction shown in the transverse field spectra (FIG. 17) is enhanced when muons enter the AuNP solution, as compared with the reference solution containing a similar surfactant concentration and evaluated under the same experimental conditions (same sample geometry and magnetic field).

μ⁺+e⁻→Mu (1)

μ⁺+H₂O→→MuH₂O⁺ (2)

e⁻_{aq}+MuH₂O⁺→Mu+H₂O (3)

H₂O+MuH₂O⁺→MuOH+H₃O⁺ (4)

Mu+e⁻_{aq}+H₂O→MuH+OH⁻ (5)

e⁻+AuNP_{aq}→AuNP⁻_{aq} (6)

Mu+C₆H₆→C₆H₆Mu* (7)

In aqueous solutions, the competition of Reactions 1-5 in the radiation track determines the muon fractions (19-21). The observed increase of the diamagnetic fraction for the AuNP solution compared with the reference surfactant solution suggests considerable electron capture by the AuNPs (Reaction 6). Wolff et al. (1970) concluded that electron-scavengers influence the reactivity of pre-solvated electrons (22) and in particular that the high concentrations of acetone and nitrate ions display considerable scavenging efficiency, whereas under acidic conditions the e⁻_{aq} yields are unchanged, indicating the proton is a weak pre-solvated electron scavenger. Computational results for electron affinities of AuNPs in solution are consistent with the positive muon (μ⁺) being less efficient at capturing pre-solvated electrons than Au/bac-14 NPs. Therefore we associate the observed increase in diamagnetic fraction to a favorable interaction between the pre-solvated electron and AuNPs at small concentrations.

The thermalization rate, v, is the inverse of the time that it takes for high energy muons to reach thermal equilibrium with the surrounding molecules. Since the reaction of solvated and presolvated electrons with AuNPs is in competition with this process (reactions 1-6) the rate of reaction of AuNPs with electrons should be at least the same as the thermalization rate (Eq. 1).

v=ke[AuNP] (Eq. 1)

where [AuNP] is the AuNP concentration calculated to be 4.89×10-7 M (see supplementary information) and the muon thermalization rate (10) is at least 1 ns−1.

The lower limit of the electron capture rate constant (ke) for the AuNPs, using Eq. 1, was determined to be 2×10¹⁵ M⁻¹ s⁻¹. This large electron capture rate constant suggests the AuNPs are reacting rapidly with the pre-solvated electrons (eₚᵣₑ⁻), which relax on the ˜75 and ˜400 femtosecond (23) timescales. The large electron capture rate constant allows for the exciting prospect of controlling sub-picosecond reactions of reactive intermediates with metal NPs.

The electron capture of AuNPs is largest at 298 K and is diminished with an increase of temperature (FIG. 17). The temperature dependence of the rate constant, as well, indicates a pre-solvated electron capture due to the strong temperature dependence of the electron thermalization/solvation process. At higher temperatures, the increased rate of solvation (2, 23) competes with the AuNP pre-solvated electron capture.

The electron capture rates are at least two or more orders of magnitude larger than those reported for other well-known scavengers, such as KNO_{B} (1.2×10¹³ M⁻¹ s⁻¹), DMSO (8.1×10¹¹ M⁻¹ s⁻¹) and isopropanol (2.3×10¹¹ M⁻¹ s⁻¹) despite significantly lower scavenger concentration (10⁻⁷M) in contrast with concentrations as high as 2 M (3).

### Example 6—Calculation of the Nanoparticle Concentration

A previously published method by Liu et. al. (24) was used to calculate the concentration of gold nanoparticles in solution. Using TEM (FIG. S1), the average core diameter of the AuNPs was determined using ImagePro Plus software; D=24.5±2 nm, the average number of gold atoms in a nano-particle was calculated according to equation S1

N=(π/6)(ρ/M)·D³ (S1)

Where ρ is the density of fcc gold, 19.3 g cm⁻³ and M is the molar mass of gold, 197 g mol⁻¹. An average core diameter of 24.5 would then result in an average number of gold atoms of 4.54×10⁵. The molar concentration of gold nanoparticle suspended in solution is then 4.89×10⁻⁷ mol L⁻¹ according to equation S2.

C=Nₜₒₜₐₗ/NVN_{A} (S2)

The molar absorptivity (extinction coefficient) was calculated according to the Beer-Lambert law, eq. S3.

Aₛₚᵣ=εbc (S3)

We report here ε=1.82×10⁸ M⁻¹ cm⁻¹ which compared well to the literature. Despite differences in the synthetic method of AuNPs used by Li et. al., as compared the novel Au/bac-14 nanoparticle.

### Example 7—Calculation of the Rate of Electron Capture

For the concentrations of AuNPs prepared from the novel one-pot synthesis outlined above, we assume a pseudo-first/first order reaction rate (20). Muon thermalization occurs on the sub nanosecond timescale. The rate of electron capture can be described by equation S4.

v=kₑ[AuNP] (S4)

Where, kₑ is the effective rate constant (M⁻¹ s⁻¹) and [AuNP] is the concentration of gold nanoparticles and v is the minimum rate of ˜1 ns⁻¹.

We calculate the effective rate constant (M⁻¹ s⁻¹) is 2.04×10¹⁵ M⁻¹ s⁻¹ for a gold nanoparticle concentration of 4.89×10⁻⁷ mol L⁻¹.

### Example 8 Calculations of the Diffusion Limit for Nanoparticle Reactions

A combined model for kinetic diffusion was used to estimate the rate of hydrated electron capture based on the modified Stokes-Einstein form for spherical (uncharged) particles through a liquid of low Reynolds number, taking into account the diffusion coefficient D for the hydrated electron. A report by Schmidt and co-workers on kinetic diffusion for hydrated electrons at ambient temperatures and atmospheric pressure, revealed that in H₂O, D=(4.90±0.02)×10⁻⁵ cm² s⁻¹ (25).

The diffusion coefficient is inversely proportional to the radius of the particle, D=k_{B}T/6πηr. Nanoparticles diffuse slower than small molecules, by roughly an order of magnitude based on the Stokes-Einstein equation. An independent report on TiO₂ nanoparticles capped with p-toluenesulfonic acid showed that D=10⁻¹⁰ m² s⁻¹ (26).

The rate constant of a diffusion-controlled reaction is given as k_{d}=4πR^{*}DN_{A}, where two reactant molecules react if they come a distance R^{*} from each other. In this case, diffusion of small molecules to the surface of a nanoparticle is faster if the particle is larger.

Given the size of the Gold nanoparticles (20 nm, 2×10⁻⁶ cm), the center-to-center distance R^{*} is close to the radius of the nanoparticle r. This simplifies the diffusion model by eliminating the dependence of D on r⁻¹, and k_{d}=4πR^{*}DN_{A}≈4πR^{*}N_{A}k_{b}T/6πηr=2N_{A}k_{b} T/3η.

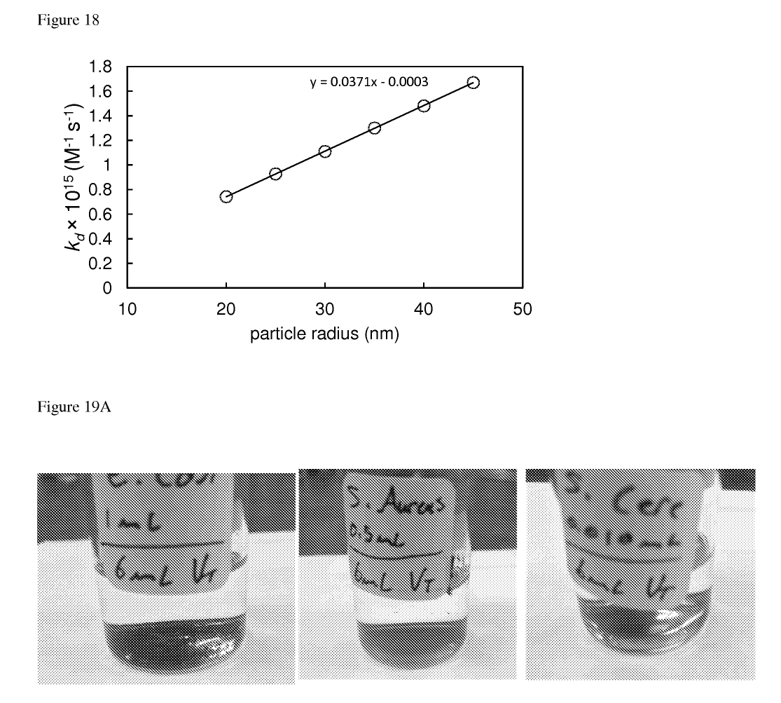

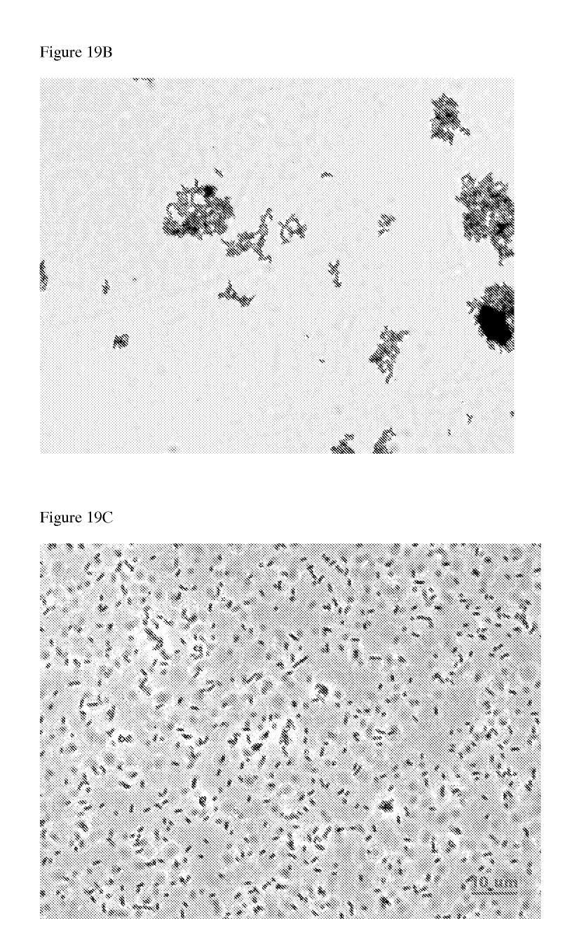

The rate of nanoparticle-electron combination should be defined by the diffusion of the hydrated electron, given that the kinetic diffusion is remarkably fast in water (25). The model predicts, k_{d}=7.42×10¹⁴ M⁻¹ s⁻¹, which is agreement with experiment. This relationship is plotted in FIG. 18.

### Tables:

TABLE 1
Summary of UV/Vis absorbance maximum peaks for gold nanoparticles
synthesized with different benzyl alkyl ammonium chloride chain lengths.

| | Surface |
|---|---|
| Surfactant | Plasmon |
| Chain | Band Peak |
| length | (nm) |
| C-12 | 526 |
| C-16 | 517 |
| C-18 | 518 |

TABLE 2
DFT Optimized Au/Bac-14 Structure Coordinates

| Atom Type | X | Y | Z |
|---|---|---|---|
| N | −3.79969100 | −1.72088100 | −0.21235500 |
| C | −3.82329500 | −3.12140600 | −0.78274700 |
| H | −4.83032100 | −3.34092100 | −1.14214100 |
| H | −3.11721500 | −3.18658300 | −1.61206400 |
| H | −3.54129200 | −3.83375000 | −0.00204300 |
| C | −4.09035800 | −0.71758500 | −1.32330200 |
| H | −5.07154600 | −0.93940700 | −1.74661800 |
| H | −4.07253100 | 0.28774700 | −0.89461900 |
| H | −3.31963400 | −0.81303400 | −2.08980300 |
| C | −2.42939600 | −1.41059400 | 0.44833300 |
| H | −2.52571600 | −0.39315700 | 0.84342700 |
| H | −2.34320100 | −2.11554400 | 1.28513800 |
| C | −1.19715800 | −1.50264400 | −0.46111300 |
| H | −1.09582300 | −2.49893300 | −0.91555500 |
| H | −1.26120100 | −0.75755500 | −1.26383500 |
| C | 0.07219900 | −1.19410500 | 0.37111800 |
| H | 0.14076400 | −1.89305400 | 1.22090100 |
| H | −0.02168800 | −0.18147700 | 0.78936700 |
| C | 1.36746100 | −1.27810900 | −0.46348000 |
| H | 1.29444100 | −0.58198000 | −1.31293100 |
| H | 1.47013000 | −2.29003100 | −0.88945500 |
| C | 2.62695700 | −0.94070900 | 0.36322000 |
| H | 2.68064300 | −1.61191300 | 1.23613600 |
| H | 2.53060900 | 0.08122900 | 0.76145800 |
| C | 3.93876100 | −1.05306700 | −0.44270700 |
| H | 3.88656100 | −0.38618700 | −1.31805500 |
| H | 4.03832700 | −2.07769100 | −0.83711000 |
| C | 5.19130400 | −0.70433000 | 0.39007600 |
| H | 5.22679700 | −1.35346600 | 1.28029000 |
| H | 5.10134700 | 0.32821300 | 0.76349300 |
| C | 6.51259700 | −0.84910200 | −0.39512400 |
| H | 6.47864100 | −0.20390000 | −1.28783500 |
| H | 6.60490600 | −1.88382100 | −0.76363300 |
| C | 7.76006700 | −0.49426400 | 0.44271800 |
| H | 7.78288000 | −1.12752500 | 1.34463400 |
| H | 7.67491700 | 0.54561000 | 0.79715600 |
| C | 9.08680600 | −0.66233200 | −0.32868000 |
| H | 9.06503000 | −0.03168700 | −1.23222500 |
| H | 9.17344600 | −1.70350300 | −0.67976200 |
| C | 10.33122400 | −0.30383600 | 0.51233600 |
| H | 10.34683400 | −0.92776800 | 1.42086600 |
| H | 10.24871300 | 0.74015700 | 0.85548600 |
| C | 11.66101100 | −0.48498800 | −0.25094800 |
| H | 11.64770500 | 0.13781500 | −1.16017000 |
| H | 11.74591500 | −1.52948900 | −0.59244000 |
| C | 12.90364300 | −0.12453500 | 0.59246500 |
| H | 12.91519800 | −0.74466400 | 1.50252200 |
| H | 12.82054100 | 0.92012500 | 0.93061400 |
| C | 14.22958400 | −0.31289600 | −0.17415400 |
| H | 14.25853800 | 0.32006400 | −1.07197800 |
| H | 15.09472800 | −0.05088400 | 0.44951800 |
| H | 14.35193100 | −1.35590300 | −0.49782500 |
| C | −4.85663000 | −1.58947000 | 0.94318300 |
| H | −4.59923200 | −2.39457900 | 1.64026900 |
| H | −4.63829800 | −0.61660400 | 1.39724400 |
| C | −6.30464800 | −1.66353700 | 0.51443200 |
| C | −7.01042900 | −0.46802600 | 0.24021600 |
| C | −6.99340800 | −2.89720800 | 0.45804600 |
| C | −8.37090700 | −0.51437000 | −0.11368100 |
| H | −6.49495900 | 0.48702800 | 0.32417600 |
| C | −8.35390700 | −2.94272500 | 0.10361600 |
| H | −6.47617500 | −3.82085000 | 0.71468500 |
| C | −9.04377400 | −1.74992300 | −0.18856400 |
| H | −8.90522700 | 0.40995200 | −0.31726000 |
| H | −8.87478300 | −3.89631000 | 0.06854200 |
| H | −10.09682000 | −1.78204100 | −0.45733000 |
| Cl | −4.06887200 | 1.79805800 | 1.17740700 |
| Au | −1.88318300 | 2.39777200 | −0.23800300 |

SCF Done: E(UB3LYP) = −1106.77707988

| | (Hartree/Particle) |
|---|---|
| Zero-point correction= | 0.619346 |
| Thermal correction to Energy= | 0.652702 |
| Thermal correction to Enthalpy= | 0.653646 |
| Thermal correction to Gibbs Free Energy= | 0.544584 |
| Sum of electronic and zero-point Energies= | −1106.157734 |
| Sum of electronic and thermal Energies= | −1106.124378 |
| Sum of electronic and thermal Enthalpies= | −1106.123434 |
| Sum of electronic and thermal Free Energies= | −1106.232496 |

TABLE 3
Calculated Isotropic Fermi Contact Coupling
between proton/muon and unpaired electron.

| | | Aᵢₛₒ | |
|---|---|---|---|
| | | (theoretical)/MHz | Aᵢₛₒ (Mu)/MHz |
| — | nuclei | B3LYP/6-31G* | B3LYP/6-31G* |
| para | ¹H (top) | 138.532 | 441 |
| | ¹H (bottom) | 139.127 | 442 |
| ortho | ¹H | 135.674- | 430 |
| meta | ¹H (top) | 142.845 | 455 |
| | ¹H (bottom) | 144.784- | 461 |
| ipso | ¹H | 128.776 | 409 |

1. A stabilized metal nanoparticle composition comprising metal nanoparticles and a non-covalently bound ligand in a stable single phase aqueous solution.

2. The composition according to claim 1, wherein the nanoparticles are monometallic, bimetallic, or polymetallic in composition.

3. The composition according to claim 1, wherein the nanoparticles are comprise gold, silver, copper, or titanium nanoparticles, or alloys thereof.

4. The composition according to claim 1 wherein the nanoparticles are gold nanoparticles.

5. The composition according to claim 1 wherein the metal nanoparticles are capped with the non-covalently bound ligand.

6. The composition according to claim 5 wherein the ligand is benzylalkyl(C8-18)ammonium chloride.

7. The composition according to any of claims 1-6 where the metal nanoparticles are capable of capturing pre-solvated electrons from a solution.

8. The composition according to any of claims 1-7, where the size of the metal nanoparticles is optionally between 1-10, 1-50, 1-100 and 1-1000 nanometers respectively.

9. A therapeutic agent for radiation therapy or radiation protection comprising the composition of claims 1-8, and optionally a biologically compatible carrier.

10. A therapeutic agent, according to claim 9, capable of capturing pre-solvated and solvated electrons, to prevent the formation of reactive oxygen and nitrogen species.

11. A therapeutic agent, according to claims 9-10, capable of radioprotective effects comprising reducing the secondary effects of radiation induced cell damage.

12. A topical composition comprising the composition of any of claims 1-8 and a carrier, wherein the composition comprises a lotion, gel, rinse, or cream.

13. The therapeutic agent of any of claims 9-11, wherein therapeutic agent is formulated for topical application and comprises a lotion, gel, rinse, or cream.

14. The therapeutic agent of any of claims 9-11 further comprising an additional antimicrobial agent.

15. The therapeutic agent of any of claims 9-11, wherein therapeutic agent further comprises protection from microbial infection.

16. A catalyst for oxidation reactions comprising the composition of any of claims 1-8.

17. The catalyst of claim 16, wherein the catalyst is capable of inhibiting radiation induced chemical reactions.

18. The catalyst according to claim 16, wherein the catalyst is capable of moderating radiation induced chemical reactions.

19. A catalyst for pre-solvated electron based chemical reactions, comprising the composition of any of claims 1-8.

20. The catalyst according to claim 19, wherein the catalyst is capable of inhibiting pre-solvated electron based chemical reactions.

21. The catalyst according to claim 19, capable of moderating pre-solvated electron based chemical reactions.

22. A catalyst for solvated electron based chemical reactions, comprised of the composition of claims 1-8.

23. The catalyst according to claim 22, capable of inhibiting solvated electron based chemical reactions.

24. The catalyst according to claim 22, capable of moderating solvated electron based chemical reactions.

25. A method for preparing a stable single aqueous phase composition comprising metal nanoparticles comprising mixing the metal nanoparticles with an aromatic alkyl ammonium halide.

26. The method of claim 25, wherein the metal nanoparticles in the single aqueous phase are stable in water for at least three months.

27. The method of claim 25, wherein the stable single aqueous phase comprises anionic and cationic components, and wherein the metal nanoparticles are catalytically active.

28. The composition according to any of claims 1-8, where the shape of the metal nanoparticles is selected from spherical, a triangle, a square, a rectangle, a rhombus, a diamond, a pyramid, and other polygonal shapes comprising at least 5, 6, 7, 8, 9, 10, 11, 12, 13, 14, 15, 16, 17, 18, 19 or 20 or more sides or faces.

29. A microbial sensor comprising the composition according to claims any of claim 1-8 or 28, wherein the metal nanoparticles are provided in a shape arrangement that provides for a change in one or more of the optical, physical, or chemical properties of the metal nanoparticles upon binding to a microbial organism.

30. An assay for detecting the presence of a microbe, wherein the assay comprises contacting a sample to be analyzed with an amount of the composition according to claims any of claim 1-8 or 28; detecting a change in at least one property of the metal nanoparticles selected from the group consisting of the optical, physical, or chemical properties of the metal nanoparticles, and wherein detecting a change in at least one property of the metal nanoparticles indicates the presence of the microbe in the sample.
